(19) United States
(12) Patent Application Publication
(10) Pub. No.: US 2009/0062326 A1
(43) Pub. Date: Mar. 5, 2009

(54) M3 MUSCARINIC RECEPTOR ANTAGONISTS FOR TREATMENT OF M3 MUSCARINIC RECEPTOR-EXPRESSING TUMORS

(76) Inventors: Eliot R. Spindel, Lake Oswego, OR (US); Harmanjatinder Sekhon, Vancouver (CA); Pingfang Song, Beaverton, OR (US)

Correspondence Address: KLARQUIST SPARKMAN, LLP, 121 SW SALMON STREET, SUITE 1600, PORTLAND, OR, 97204, US

(21) Appl. No.: 12/281,976

(22) PCT Filed: Mar. 16, 2007
(86) PCT No.: PCT/US2007/006658

§ 371 Date: Sep. 5, 2008

Related U.S. Application Data
(60) Provisional application No. 60/783,461, filed on Mar. 17, 2006.

(51) Int. Cl. A61K 31/46; A61K 31/40; A61K 31/445; A61K 31/045; A61P 35/00; A61K 31/4725; A61K 31/216

(52) U.S. Cl. 514/291; 514/422; 514/315; 514/738; 514/305; 514/534

(57) ABSTRACT

The present disclosure provides methods of treating a tumor using M3 muscarininc receptor antagonists, such as darifenacin. In some examples, the tumor expresses M3 muscarinic receptors, such as tumors associated with smoking. Also provided are compositions that can be used to practice such methods.

### CROSS-REFERENCE TO RELATED APPLICATION

This application claims the benefit of U.S. Provisional Application No. 60/783,461 filed Mar. 17, 2066, herein incorporated by reference.

### ACKNOWLEDGMENT OF GOVERNMENT SUPPORT

This work was supported by National Institutes of Health grant numbers HD/HL 37131, CA 69533 and RR00163. Therefore, the Government of the United States of America may have certain rights in this application.

### FIELD

This application relates to methods of using a M3 muscarinic receptor antagonist (for example darifenacin) to treat a subject having a tumor, such as a tumor that expresses M3 muscarimic receptors, or is at risk for developing such a tumor, for example a lung cancer or other tumor associated with smoking. Also provided are compositions that can be used with such methods.

### BACKGROUND

Cigarette smoking over a prolonged period of time is the most important risk factor in the development of lung and other smoking related cancers. Other risk factors include exposure to passive-smoking, certain industrial substances such as arsenic, some organic chemicals, radon and asbestosis, ingestion of alcohol, radiation exposure, air pollution and tuberculosis. Many of these factors greatly increase the risk of developing lung and other smoking related cancers (such as cancer of the oropharynx, esophagus, bladder, pancreas, and cervix).

Annual global lung cancer deaths exceeded 1,000,000 in the year-2000 and are expected to exceed 2,000,000 by the year 2020 or 2030 (Proctor, Nature Rev. Cancer 1:82-6, 2002). Lung cancers are classified as either small cell lung carcinoma (SCLC) or non-small cell lung carcinoma (NSCLC) with small cell lung carcinoma (SCLC) accounting for 15-20% of primary lung cancer. Non-small cell lung carcinoma accounts for the remaining cases with squamous cell and adenocarcinoma as the most common types of NSCLC.

The five-year survival rate among-all lung cancer patients, regardless of the stage of disease at diagnosis, is only 13%. This contrasts with a five-year survival rate of 46% among cases detected while the disease is still localized. However, only 16% of lung cancers are discovered before the disease, has spread. Early detection is difficult since clinical symptoms are often not evident until the disease has reached an advanced stage. Treatment regimens are determined by the type and stage of the cancer, and include surgery, radiation therapy and/or chemotherapy.

In spite of considerable research into therapies, cancers associated with smoking, such as lung cancer, remain difficult to treat effectively. Accordingly, there is a need for improved methods for treating (including preventing or otherwise reducing the development of) such cancers.

### SUMMARY

Darifenacin is an M3 muscarinic receptor antagonist currently indicated for the treatment of urinary incontinence and irritable bowel syndrome (for example see U.S. Pat. No. 6,106,864). Surprisingly, it is shown herein that darifenacin is also useful for the treatment of cancer, such as treatment of a tumor that expresses M3 muscarinic receptors (for example lung cancer). Based on the observation that darifenacin can be used to treat cancer in a mammal in vivo, a new use for darifenacin or a pharmaceutically acceptable derivative thereof is disclosed, namely the treatment of tumors (such as cancers), for example those that expresses M3 muscarinic receptors. One particular example of tumors that can expresses M3 muscarinic receptors are tumors associated with smoking.

Provided herein are methods of treating a mammalian subject having a tumor, such as a tumor that expresses M3 muscarinic receptors or is at an increased risk for developing a tumor (such as one that expresses M3 muscarinic receptors).

Examples of tumors that expresses M3 muscarinic receptors include those associated with smoking, such as lung cancer (including small cell lung carcinoma), oral cancer, pharyngeal cancer, esophageal cancer, bladder cancer, pancreatic cancer, and cervical cancer. In particular examples, the subject who is at risk for developing a tumor is a smoker (including subjects who currently smoke regularly, or who did so for a significant amount of time in the past, such as smoked regularly for at least 10 years). However, in some examples, a subject having a tumor associated with smoking is a non-smoker (such as a subject who has never smoked in the past).

Treatment can include preventing or delaying development of a tumor, for example a tumor that expresses M3 muscarinic receptors (such as those associated with smoking), or preventing or delaying metastasis of a tumor, as well as reducing the size or volume of a tumor, and increasing the survival time of a subject having a tumor. In particular examples, the method includes administering to the subject a therapeutically effective amount of an M3 muscarinic receptor antagonist, such as darifenacin, solifenacin, zamifenacin (or a pharmaceutically acceptable derivative of any of these), thereby treating the tumor. Methods of administration include those known in the art, such as oral (for example via a tablet, such those disclosed in Example 3 of U.S. Pat. No. 6,106,864), nasal (for example via a nebulizer or inhaler), or injection.

In some examples, the method also includes determining whether a tumor in a subject expresses M3 muscarinic receptors, nicotinic receptors, or both.

The disclosed therapies can be administered alone or in combination with other medical or surgical therapies. For example, in addition to administration of darifenacin or a pharmaceutically acceptable derivative thereof to the subject (or other M3 muscarnic receptor antagonist), the subject can also receive one or more additional therapeutic agents, such as a therapeutically effective amount of anti-neoplastic chemotherapeutic agents, antibodies, radiological agents, nicotinic receptor antagonists, or combinations thereof. Such therapies can be administered before, during, or after administration of darifenacin or a pharmaceutically acceptable derivative thereof. In one example, the tumor is surgically excised or debulked prior to administration of darifenacin or a pharmaceutically acceptable derivative thereof.

Also provided by this disclosure are compositions that include a therapeutic amount of darifenacin (or other M3 muscarinic receptor antagonist), or a pharmaceutically acceptable derivative thereof, for use in the treatment of a tumor in a human, for example a tumor that expresses M3 muscarinic receptors. In particular examples, such compositions also include a therapeutic amount of one or more anti-neoplastic chemotherapeutic agents. In another particular example, such compositions further include a therapeutic amount of one or more nicotinic receptor antagonists (such as mecamylamine).

The foregoing and other objects, features, and advantages of the disclosure will become more apparent from the following detailed description, which proceeds with reference to the accompanying figures.

### BRIEF DESCRIPTION OF THE DRAWINGS

FIGS. 1A-E are bar graphs showing the affect 0-10 mM of darifenacin (bars from left to right are 0, 1 nM, 10 nM, 100 nM, 1 μM and 10 μM) on the proliferation of five SCLC cell lines (A) H187, (B) H1048, (C) H1836, (D) H1876, and (E) H520. * p<0.05 compared to control at same time point.

FIG. 2 is a graph showing the effect of darifenacin on nude mouse tumor size. Nude mice were injected with H82 cells on day 0, then treated from weeks 1-5 with the dose (mg/kg/day) darifenacin shown. Tumor volume was determined weekly by measuring with calipers (volume=height×width×depth).

FIG. 3 is a bar graph showing the effect of darifenacin on nude mouse tumor weight. Nude mice were injected with H82 cells on day 0, then treated from weeks 1-5 with the dose (mg/kg/day) darifenacin shown. At the end of 5 weeks, tumors were removed and weighed.

FIG. 4 is a bar graph showing the relation of nude mouse darifenacin dose to concentration of darifenacin achieved in mouse blood. Dashed lines show concentration of darifenacin obtained in humans treated with 30 mg extended release darifenacin per day.

FIG. 5 is a bar graph showing the effect of the non-selective muscarinic antagonist atropine on nude mouse tumor weight. Nude mice were injected with H82 cells on day 0, then treated from weeks 1-5 with the 10 mg/kg atropine. At the end of 5 weeks, tumors were removed and weighed.

FIG. 6 is a bar graph showing that nicotine stimulates acetylcholine secretion from cultured SCLC cells. H82 SCLC cells were incubated with nicotine for the times shown and secretion of ACh into the medium was measured.

### DETAILED DESCRIPTION

### Abbreviations and Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a pharmaceutically acceptable carrier” includes single or plural pharmaceutically acceptable carriers and is considered equivalent to the phrase “comprising at least one pharmaceutically acceptable carrier.” The term “or” refers to a single element of stated alternative elements or a combination of two or more elements, unless the context clearly indicates otherwise. As used herein, “comprises” means “includes.” Thus, “comprising M3 muscarinic receptors or nicotinic receptors” means “including one or more M3 muscarinic receptors, including one or nicotinic receptors, or including one or more M3 muscarinic receptors and one or more nicotinic receptors,” without excluding additional elements.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

| | |
|---|---|
| ACh | acetylcholine |
| ChAT | choline acetyltransferase |
| mAChR | muscarinic receptor |
| nAChR | nicotinic acetylcholine receptor |
| SCLC | small cell lung carcinoma |

Acetylcholine (ACh): An ester of acetic acid and choline with chemical formula CH₃COOCH₂CH₂N⁺(CH₃)₃, which can function as a chemical transmitter in both the peripheral nervous system and central nervous system. ACh is also widely synthesized by a variety of non-neuronal cell types including keratinocytes, airway epithelial cells, glia, vascular endothelium, placental trophoblasts, and ovarian follicular cells among others.

ACh is a ligand for muscarinic receptor and nicotinic receptors. The interaction of acetylcholine with the M3 receptor on cancerous or precancerous tissue can stimulate the tumor or tissue to grow. Acetylcholine can be synthesized by the tumor itself, in which case it is acting as an autocrine growth factor, or it can be delivered by local circulation in which case it is acting as a paracrine growth factor. The interaction of acetylcholine with nicotinic receptors can also stimulate tumor growth.

In particular examples, expression of ACh can be demonstrated by the presence of choline acetyltransferase (ChAT), the enzyme that synthesizes ACh, by demonstration of ACh synthesis (for example as measured by HPLC), and other methods known in the art.

Administration: To provide or give a subject an agent, such as a composition that includes an M3 muscarinic receptor antagonist (for example darifenacin or a pharmaceutically acceptable derivative thereof), by any effective route. Exemplary routes of administration include, but are not limited to, oral, injection (such as subcutaneous, intramuscular, intradermal, intraperitoneal, and intravenous), sublingual, rectal, transdermal, intranasal, intraocular, and inhalation routes.

Cancer: Malignant neoplasm that has undergone characteristic anaplasia with loss of differentiation, increased rate of growth, invasion of surrounding tissue, and is capable of metastasis.

Cancer or tumor associated with smoking: A tumor or cancer that occurs at an increased frequency in smokers, as compared to non-smokers. Particular non-limiting examples include cancers of the lung (such as small cell lung carcinomas), upper airway primary or secondary, bladder, kidneys, pancreas, mouth, throat, pharynx, larynx, esophagus, liver, kidney, lymph node, pancreas, blood cells, colon, stomach, cervix, bone marrow and blood.

Chemotherapeutic agent: In cancer treatment, refers to the administration of one or a combination of compounds to kill or slow the reproduction of rapidly multiplying cells. Chemotherapeutic agents include anti-neoplastics known by those skilled in the art, including, but not limited to: 5-fluorouracil (5-FU), azathioprine, cyclophosphamide, antimetabolites (such as Fludarabine), antineoplastics (such as Etoposide, Doxorubicin, methotrexate, and Vincristine), carboplatin, cis-platinum and the taxanes, such as taxol, monoclonal antibodies such as Avastin or Herceptin, and growth pathway inhibitors such as Gleevac. In particular examples, such chemotherapeutic agents are administered in combination with darifenacin therapy (for example before, during or after administration of a therapeutic amount of darifenacin).

Choline acetyltransferase (ChAT): The enzyme that makes acetylcholine.

Darifenacin: (S)-2-{1-[2-(2,3-dihydrobenzofuran-5-yl)ethyl]-3-pyr-rolidinyl}-2,2-diphenyl-acetamide, including pharmaceutically acceptable derivatives thereof which act as a selective antagonist of the M3 muscarinic receptor (relative to the M1 and M2 muscarinic receptor subtypes).

The chemical structure of darifenacin and derivatives thereof, which can be used in the treatments disclosed herein, are provided in U.S. Pat. Nos. 5,233,053 and 6,106,864 (herein incorporated by reference) and in European Patent No 0388054, Examples 1B and 8 (it is referred to therein as 3-(S)-(G)-(1-carbamoyl-1,1-diphenylmethyl)-1-[2-(2,3-dihydro-benzofuran-5-yl)ethyl]pyrrolidine).

Exemplary dosage and administration regimens for human subjects are known in the art, and can include darifenacin at doses of 3.75-40 mg daily (for a 70 kg person), such as 7.5-30 mg daily, for example 7.5 or 15 mg once daily (q.d.) by mouth.

Increased risk: An elevated likelihood that a certain event will occur. For example, smoking is an attribute that is associated with an increased probability of the development of lung cancer.

Lung cancer: A malignant tumor of the lungs, such as a bronchogenic carcinoma.

The World Health Organization classifies lung cancer into four major histological types: (1) squamous cell carcinoma (SCC), (2) adenocarcinoma, (3) large cell carcinoma, and (4) small cell lung carcinoma (SCLC). (The World Health Organization, “The World Health Organization histological typing of lung tumours,” Am J Clin Pathol 1982; 77:123-136). However, there is a great-deal of tumor heterogeneity even within the various subtypes, and it is not uncommon for lung cancer to have features of more than one morphologic subtype. The term non-small cell lung carcinoma (NSCLC) includes squamous, adenocarcinoma and large cell carcinomas.

Muscarinic receptor (mAChR): Membrane-bound G-protein coupled acetylcholine receptors that are more sensitive to muscarine than to nicotine. Muscarinic receptors are selectively activated by muscarine and blocked by atropine.

Five mAChR subtypes have been identified, originally designated m1, m2, m3, m4 and m5. These structurally distinct subtypes have characteristic distributions, pharmacological (binding) profiles and physiological functions. In many tissues/cells, multiple subtypes of mAChR coexist, with each of them playing a role in parasympathetic innervation.

M3 muscarinic receptor: The M3 muscarinic acetylcholine receptor (M3 mAChR) triggers contraction of smooth muscle cells through an interaction with G_{q} proteins to stimulate phosphoinositide hydrolysis and mobilize Ca²⁺.

The M3 mAChRs are located at many places in the body. They are located in the smooth muscles of the blood vessels, as well as in the lungs (for vasodilation and bronchoconstriction). They are also in the smooth muscles of the gastrointestinal tract, which help in increasing intestinal motility and dilating sphincters. The M3 receptors are also located in many glands which help to stimulate secretion in salivary glands and other glands.

The term M3 muscarinic receptor includes any M3 mAChR gene, cDNA, mRNA, or protein, such as one that is sensitive to the M3 mAChR antagonist darifenacin. It is shown herein that substantially reducing the biological activity of the M3 mAChR by contact with the M3 mAChR antagonist darifenacin can be used to reduce the growth or even kill an M3 mAChR-expressing tumor cell. Selective M3 muscarinic receptor antagonists have greater affinity for the M3 mAChR than the M1, M2; M4 and M5 mAChRs (such as an affinity at least 5-times greater for the M3 mAChR, than the M1, M2, M4 and M5 mAChRs). Particular examples of such antagonists include 4-diphenylacetoxy-N-methylpiperidine methiodide (4-DAMP); hexahydro-sila-difenidol hydrochloride, p-fluoro analog (p-F-HHSiD); darifenacin; solifenacin; zamifenacin; and oxybutynin. Selective M3 muscarinc antagonists also include such compounds as tiotropium (trade name Spiriva), which achieve selectivity by longer association with the M3 receptor than the other muscarinic receptor subtypes.

M3 mAChR sequences are publicly available. For example, GenBank Accession Nos: NM_{—}000740 and NP_{—}000731 disclose exemplary human m3 muscarinic receptor nucleic acid and protein sequences, respectively and Genank Accession Nos: M16408 and AAA41553 disclose exemplary rat M3 mAChR nucleic acid and protein sequences, respectively. However, one skilled in the art will appreciate that a M3 mAChR sequence can include allelic variants, variants, fragments, homologs or fusion sequences that retain the ability to trigger contraction of smooth muscle cells through an interaction with G_{q} proteins to stimulate phosphoinositide hydrolysis and mobilize Ca²⁺.

Malignant: Cells which have the properties of anaplasia invasion and metastasis.

Neoplasm: Abnormal growth of cells.

Nicotinic receptor: The nicotinic acetylcholine receptors (nAChRs) are diverse members of the neurotransmitter-gated ion channel superfamily, which play neuromodulatory roles in the central nervous system. Binding of ACh to nAChRs allows entry of sodium or calcium into the cell. The endogenous neurotransmitter for nAChRs is acetylcholine, but they are also opened by nicotine.

Nicotinic acetylcholine receptors are present in many tissues in the body. The neuronal receptors are found in the central nervous system and the peripheral nervous system. The neuromuscular receptors are found in the neuromuscular junctions of somatic muscles; stimulation of these receptors causes muscular contraction.

The term nicotinic receptor includes any nicotinic receptor gene, cDNA, mRNA, or protein, such as one that is sensitive to acetylcholine and nicotine. There are two general families of central nAChRs: (i) high-affinity (β2 subunit-containing nAChRs, which exist in a heteropentameric configuration of α subunits combined with β subunits) receptors, which are sensitive to the nicotinic antagonists mecamylamine and dihydro-β-erythrodine and (ii) low-affinity (α7 subunit-containing nAChR homopentameric complexes) receptors which are sensitive to the snake venom toxin α-bungarotoxin and the selective antagonist methyllycaconitine. Therefore, wherein referring to detection or expression of “nicotinic receptors”, this includes detection or expression of the alpha or beta subunits of the nAchR, such as the α3, α5, α7, β2, and β4 subunits. Selective nicotinic receptor antagonists include Inversine® (mecamylamine HCl), methyllycaconitine, and dihydro-β-erythrodine.

Nicotinic receptor sequences are publicly available. For example, GenBank Accession Nos: NM_{—}000748 and NP_{—}000739 disclose exemplary human nicotinic receptor nucleic acid and protein sequences, respectively and GenBank Accession Nos: NM_{—}009602 and NP_{—}033732 disclose exemplary mouse nicotinic receptor nucleic acid and protein sequences, respectively. However, one skilled in the art will appreciate that a nicotinic receptor sequence can include allelic variants, variants, fragments, homologs or fusion sequences that retain the ability to open in the presence of acetylcholine or nicotine.

Normal cells: Non-tumor, non-malignant cells.

Pharmaceutically Acceptable Carrier: Compositions or formulations suitable for pharmaceutical delivery of one or more therapeutic molecules, such as darifenacin, to a subject. The pharmaceutically acceptable carriers (vehicles) useful in this disclosure are conventional (for example see Remington's Pharmaceutical Sciences, by E. W. Martin, Mack Publishing Co., Easton, Pa., 19th Edition (1995)). In general, the nature of the carrier will depend on the particular mode of administration being employed. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate, sodium lactate, potassium chloride, calcium chloride, and triethanolamine oleate.

Radiological agent: In cancer treatment, refers to the administration of one or a combination of radioactive compounds to damage the DNA of cells, thereby killing or slowing the reproduction of rapidly multiplying cells. Exemplary methods of administering radiological agents to a subject include external beam radiotherapy (XBRT) or teletherapy, brachytherapy or sealed source radiotherapy, and unsealed source radiotherapy. The radiological agents that can be administered to a subject in combination with the disclosed therapies that include darifenacin, include those known by those skilled in the art, including, but not limited to: ionizing radiation (such as x-rays and gamma rays).

Sample: Includes biological samples that contain cells, genomic DNA, RNA, or proteins (or combinations thereof) obtained from a subject, such as those present in peripheral blood, urine, saliva, sputum, tissue biopsy, surgical specimen, fine needle aspirate, and autopsy material. In a particular example, a sample is a biological sample obtained from a tumor in a subject.

Smoker: A human who smokes or chews tobacco-containing products, such as cigars, cigarettes, pipes, or chew, on a regular basis (such as at least once daily). Also includes humans who previously regularly smoked for a period of at least 10 years.

Subject: Living multi-cellular organisms, a category that includes human and non-human mammals, as well as other veterinary subjects such as non-human primates, cows, cats, horses, mice, rats, rabbits, and dogs.

Therapeutically effective amount: An amount of a therapeutic agent (such as a composition that includes darifenacin or a pharmaceutically acceptable derivative thereof (or other M3 mAChR antagonist), that alone, or together with one or more additional therapeutic agents, induces the desired response, such as treatment of a tumor that expresses M3 mAChRs or both M3 mAChRs and nicotinic receptors, such as a tumor associated with smoking. In one example, it is an amount of darifenacin needed to prevent or delay the development of a tumor, prevent or delay the metastasis of a tumor, cause regression of an existing tumor, or treat one or more signs or symptoms associated with a tumor, in a subject. Ideally, a therapeutically effective amount provides a therapeutic effect without causing a substantial cytotoxic effect in the subject. The preparations disclosed herein are administered in therapeutically effective amounts.

In one example, a desired response is to decrease the size, volume, or number of tumors, for example tumors that express M3 mAChRs (such as a tumor associated with smoking). For example, the composition that includes darifenacin (or other M3 mAChR antagonist) can in some examples decrease the size, volume, or number of tumors that express M3 mAChRs by at least 5%, at least 10%, at least 15%, at least 20%, at least 25%, at least 30%, at least 50%, at least 75%, at least 90%, or even 100% as compared to a response in the absence of the therapeutic composition.

In general, an effective amount of a composition that includes darifenacin (or other M3 mAChR antagonist) administered to a human or veterinary subject will vary depending upon a number of factors associated with that subject, for example the overall health of the subject. An effective amount of a composition that includes darifenacin can be determined by varying the dosage of the product and measuring the resulting therapeutic response, such as the regression of a tumor. Effective amounts also can be determined through various in vitro, in vivo or in situ immunoassays. The disclosed therapeutic agents can be administered in a single dose, or in several doses, as needed to obtain the desired response. However, the effective amount of can be dependent on the source applied, the subject being treated, the severity and type of the condition being treated, and the manner of administration.

In particular examples, a therapeutically effective dose of darifenacin (for example as a hydrobromide salt) includes 7.5-40 mg daily (such as 7.5-30 mg) of darifenacin presented in slow release matrix tablets (see Example 3 of U.S. Pat. No. 6,106,864, herein incorporated by reference), such as 7.5 mg, 15 mg, or 30 mg of such tablets daily. In particular examples, such daily dosages are administered in one or more divided doses (such as 2, 3, or 4 doses) or in a single formulation.

In a particular example, a therapeutically effective dose of solifenacin (for example as solifenacin succinate, such as that sold as VESIcare®) includes 5-20 mg daily (such as 5 or 10 mg daily) administered orally. In another particular example, a therapeutically effective dose of zamifenacin includes 5-100 mg daily (such as 30 mg twice daily) administered orally. In a particular example, a therapeutically effective dose of oxybutynin (for example as oxybutynin chloride, such as that sold as DITROPAN XL®) includes 2.5-30 mg daily (such as 2.5, 5, or 10 mg once daily, or 5 mg two, three or four times daily) administered orally. In a particular example, a therapeutically effective dose of 4-DAMP includes 5-100 μg/kg daily (such as 10-30 μg/kg daily) administered intravenously. In a particular example a therapeutically effective dose of tiotropium bromide (for example such as that sold as Spriva) includes at least 0.5 μg administered by inhalation once daily, such as at least 1 μg, at least 5 μg, at least 10 μg, or at least 18 μg administered by inhalation once daily, for example 1-20 μg administered by inhalation once daily. In a particular example a therapeutically effective dose of tiotropium bromide (for example such as that sold as Spriva) includes 18 μg administered by inhalation once daily.

The disclosed compositions that include darifenacin (or other mAChR antagonist) can be administered alone, in the presence of a pharmaceutically acceptable carrier, in the presence of other therapeutic agents (such as a chemotherapeutic), or both.

Treating or treatment: Refers to a therapeutic intervention that ameliorates a sign or symptom of a disease or pathological condition related to a disease (such as a tumor, for example a tumor that expresses M3 mAChRs, for example lung cancer, cervical cancer, or pancreatic cancer). Treatment can also induce remission or cure of a condition, such as a tumor. In particular examples, treatment includes preventing a disease, for example by inhibiting the full development of a disease, such as preventing development of a tumor (such as a metastasis or the development of a primary tumor in a smoker). Prevention does not require a total absence of a tumor.

Reducing a sign or symptom associated with a tumor (such as a tumor associated with smoking) can be evidenced, for example, by a delayed onset of clinical symptoms of the disease in a susceptible subject (such as a smoker who has not yet developed a tumor), a reduction in severity of some or all clinical symptoms of the disease, a slower progression of the disease (for example by prolonging the life of a subject having tumor), a reduction in the number of relapses of the disease, an improvement in the overall health or well-being of the subject, or by other parameters well known in the art that are specific to the particular tumor. In a specific example; treatment of a tumor decrease the size, volume, or number of tumors (such as a tumor that express M3 mAChRs, nicotinic receptors, or both) by at least 5%, at least 10%, at least 15%, at least 20%, at least 25%, at least 30%, at least 50%, at least 75%, at least 90%, or even 100%.

Tumor: A neoplasm.

Under conditions sufficient for: A phrase that is used to describe any environment that permits the desired activity;

In one example, includes administering a therapeutically effective amount of a composition that includes darifenacin (or other M3 mAChR antagonist) sufficient to allow the desired activity. In particular examples the desired activity is treatment of a tumor, such as a tumor that expresses M3 mAChRs, for example a tumor associated with smoking.

Unit dose: A physically discrete unit containing a predetermined quantity of an active material calculated to individually or collectively produce a desired effect, such as a therapeutic effect. A single unit dose or a plurality of unit doses can be used to provide the desired effect, such as treatment of a tumor, for example a tumor that expresses M3 mAChRs. In one example, a unit dose includes a desired amount of darifenacin, such as 7.5 mg-30 mg of darifenacin as a hydrobromide salt in a slow release tablet.

### Methods of Treatment

Methods are provided for treating a mammalian subject having a tumor or who is at an increased risk for developing a tumor, such as a tumor that expresses M3 muscarinic receptors (M3 mAChRs). In particular examples, the tumor expresses M3 muscarinic receptors and nicotinic receptors. In some examples, the subject is a human subject who is treated in vivo. For example, the human subject can be a smoker having or at risk for developing a tumor that expresses M3 mAChRs, a non-smoker having a tumor that expresses M3 mAChRs (such as a tumor associated with smoking, such as lung cancer), or a non-smoker having an increased risk for developing a tumor that expresses M3 muscarinic receptors.

In particular examples, the method includes administering to the subject a therapeutically effective amount of an M3 mAChR antagonist, thereby treating the tumor. One particular example of an M3 mAChR antagonist is darifenacin (including pharmaceutically acceptable derivatives thereof). The structure of darifenacin is shown below:

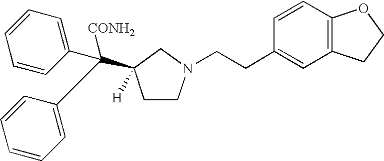

Darifenacin can act as a selective antagonist of the M3 mAChR (relative to the M1 and M2 mAChR subtypes), and is currently indicated for the treatment of urinary incontinence and irritable bowel syndrome. However, it is demonstrated herein that therapeutically effective amounts of darifenacin can be used to treat tumors, for example tumors that express M3 mAChRs. Exemplary pharmaceutically acceptable derivatives of darifenacin are provided in U.S. Pat. Nos. 6,106,864 and 5,233,053 and EP Patent No. 0388054 (all herein incorporated by reference). Such pharmaceutically acceptable derivatives of darifenacin can also be used in the methods and compositions disclosed herein, for example for the treatment of a tumor that express M3 mAChR. However, other M3 mAChR antagonists can also be used, such as solifenacin, zamifenacin, 4-DAMP, or oxybutynin.

Treatment of a tumor, such as one that express M3 mAChRs (and in some examples also expresses nicotinic receptors) can include preventing or delaying the development of the tumor in a subject (such as preventing metastasis or the development of a primary tumor), and can also include reducing signs or symptoms associated with the presence of such a tumor (for example by reducing the size or volume of the tumor). However, treatment does not require 100% inhibition of tumor growth or a 100% reduction in the tumor. In a specific example, treatment includes reducing the growth of cells of the tumor that express M3 muscarinic receptors in the subject, or even kill the tumor cells (for example by causing the cells to undergo apoptosis). For example, treatment can include reducing the size or volume (or both) of an M3 muscarinic receptor-expressing tumor in the subject by at least 5%, at least 10%, at least 25%, at least 50%, at least 75%, at least 80%, at least 90%, or even at least 100%. In particular examples, such reduced size or volume can occur in at least 3 weeks, at least 4 weeks, at least 5 weeks, at least 8 weeks, or even at least 12 weeks. Such reduced tumor growth can in some examples decrease or slow metastasis of the tumor, or reduce the size or volume of the tumor. In some examples, treatment using the methods disclosed herein prolongs the time of survival of the subject.

Tumors that can be Treated

Methods are disclosed herein for treating tumors, such as those that express M3 mAChRs (and in some example also express ChAT, nicotinic receptors, or combinations thereof). A tumor is an abnormal growth of tissue that results from excessive cell division. A particular example of a tumor is cancer.

In one example, the current application is useful for the treatment (such as the prevention) of tumors (such as cancers) that express M3 mAChRs, for example, those associated with smoking. Tumors associated with smoking include those that occur at a higher rate in-persons who currently regularly smoke (including chew tobacco products), or who regularly smoked in the past, for example smoked regularly for at least 10 years. However, such tumors also occur in non-smokers; including subjects who have never smoked. As such, non-smokers as well as smokers can be treated using the methods disclosed herein. Examples of tumors associated with smoking include, but are not limited to: lung cancer as well as cancer of the bladder, cervix, kidneys, pancreas, and cancer of the upper airways including cancer of the mouth, throat, pharynx, larynx, or esophagus.

In one example, the tumor that expresses M3 mAChRs is a lung cancer, which can be defined by a number of histologic classifications including: squamous cell carcinomas such as squamous carcinoma; small cell carcinomas such as oat cell carcinoma, intermediate cell type carcinoma, combined oat and cell carcinoma; adenocarcinomas such as acinar adenocarcinoma, papillary adenocarcinoma, bronchioloalveolar carcinoma, and solid carcinoma with mucus formation; large cell carcinoma such as giant cell carcinoma and clear cell carcinoma; adenosquamous carcinoma; carcinoid; and bronchial gland carcinomas such as adenoid cystic, and mucoepidermoid carcinoma. In a particular example, the subject to be treated has a SCLC (or has had an SCLC removed surgically or by other routine methods).

In another example, tumors that produce acetylcholine, the ligand for muscarinic and nicotinic receptors, can be particularly sensitive to darifenacin (or other M3 muscarinic receptor antagonist) because there are more growth stimulatory factors present that can be blocked.

Carcinoma, especially small cell carcinoma of the lung, has the ability to metastasize early and widely, initially to lymph nodes, then to brain, bone, liver, and skin. In particular examples, the disclosed methods can be used to treat (such as prevent or delay onset of) such metastasis.

### Administration

Methods of administration of the disclosed therapeutic agents are routine, and can be determined by a skilled clinician. For example, the disclosed therapies (such as those that include darifenacin or a pharmaceutically acceptable derivative thereof, or other M3 mAChR antagonists) can be administered orally, topically, transdermally, parenterally, or via inhalation or spray. In a particular example, darifenacin (or other M3 muscarinic receptor antagonist) is administered orally to a mammalian subject, such as a human. In another example, darifenacin (or other M3 mAChR antagonist) is administered via inhalation to a mammalian subject, such as a human having lung cancer, for example via a nebulizer or cigarette.

The therapeutic compositions, such as those that include darifenacin, can further include biologically active or inactive compounds (or both), such as anti-neoplastic chemotherapeutic agents and conventional non-toxic pharmaceutically acceptable carriers, respectively.

In a particular example, a therapeutic composition that includes a therapeutically effective amount of darifenacin (or other M3 mAChR antagonist) further includes biologically inactive compounds. Examples of such biologically inactive compounds include, but are not limited to: carriers, thickeners, diluents, buffers, preservatives, and carriers. The pharmaceutically acceptable carriers useful for these formulations are conventional (see Remington's Pharmaceutical Sciences, by E. W. Martin, Mack Publishing Co., Easton, Pa., 19th Edition (1995)). In general, the nature of the carrier will depend on the particular mode of administration being employed. For instance, parenteral formulations can include injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can include minor amounts of non-toxic auxiliary substances, such as wetting or emulsifying agents, preservatives, and pH buffering agents and the like, for example sodium acetate or sorbitan monolaurate.

In a particular example, a therapeutic composition that includes a therapeutically effective amount of darifenacin (or other M3 mAChR antagonist) further includes therapeutically effective amounts of one or more other biologically active compounds. Examples of biologically active compounds include, but are not limited to: nicotinic receptor antagonists (such as mecamylamine HCl), anti-neoplastic chemotherapeutic agents, antibiotics, alkylating agents, antioxidants, other M3 mAChR antagonists, and so forth (such as those listed below under “additional treatments”). However, one skilled in the art will appreciate that darifenacin (or other M3 surgically antagonists) and the other biologically active compounds can also be administered separately (instead of in a single composition).

The therapeutically effective amount of the compound or compounds administered can vary depending upon the desired effects and the subject to be treated. In one example, darifenacin is orally administered to a human in the form of its hydrobromide salt. For example, a human subject can be orally administered darifenacin or a pharmaceutically acceptable derivative thereof in a dosage form that is adapted to release at least 10% of the darifenacin or the pharmaceutically acceptable derivative thereof in the lower gastrointestinal tract of the subject. A particular example of such a formulation is the slow release matrix tablets described in U.S. Pat. No. 6,106,864 (Example 3, herein incorporated by reference).

In one example, the method includes daily oral administration of about 7.5 mg to 40 mg darifenacin (or a pharmaceutically acceptable derivative thereof) to the subject (such as a human subject). For example, a human can be orally administered 7.5 mg to 40 mg darifenacin (or a pharmaceutically acceptable derivative thereof) daily, such as 7.5 mg to 30 mg daily, 7.5 mg to 20 mg daily, for example 7.5 mg daily, 15 mg daily, or 20 mg daily. The dosage can be administered in divided doses (such as 2, 3, or 4 divided doses per day), or in a single dosage daily. In particular examples, the subject is administered the therapeutic composition that includes darifenacin on a multiple daily dosing schedule, such as at least two consecutive days, 10 consecutive days, and so forth, for example for a period of weeks, months, or years. In one example, the subject is administered the therapeutic composition that includes darifenacin daily for a period of at least 30 days, such as at least 2 months, at least 4 months, at least 6 months, at least 12 months, at least 24 months, or at least 36 months.

### Additional Treatments

In particular examples, prior to, during, or following, administration of a therapeutic amount of M3 mAChR antagonist (such as darifenacin or a pharmaceutically acceptable derivative thereof), the subject can receive one or more other therapies. In one example, the subject receives one or more treatments to remove or reduce the tumor prior to administration of a therapeutic amount of M3 mAChR antagonist (such as darifenacin or a pharmaceutically acceptable derivative thereof).

Examples of such therapies include, but are not limited to, surgical treatment for removal of the tumor, as well as anti-tumor pharmaceutical treatments which can include radiotherapeutic agents, anti-neoplastic chemotherapeutic agents, antibiotics, tumor-specific antibodies, alkylating agents and antioxidants, kinase inhibitors, and other agents. Particular examples of additional therapeutic agents can that can be used include microtubule binding agents, DNA intercalators or cross-linkers, DNA synthesis inhibitors, DNA and/or RNA transcription inhibitors, antibodies, enzymes, enzyme inhibitors, gene regulators, angiogenesis inhibitors, and nicotinic receptor antagonists. These agents (which are administered at a therapeutically effective amount) and treatments can be used alone or in combination. Methods and therapeutic dosages of such agents are known to those skilled in the art and can be determined by a skilled clinician.

“Microtubule-binding agent” refers to an agent that interacts with tubulin to stabilize or destabilize microtubule formation thereby inhibiting cell division. Examples of microtubule binding agents that can be used in conjunction with darifenacin therapy include, without limitation, paclitaxel, docetaxel, vinblastine, vindesine, vinorelbine (navelbine), the epothilones, colchicine, dolastatin 15, nocodazole, podophyllotoxin and rhizoxin. Analogs and derivatives of such compounds also can be used and are known to those of ordinary skill in the art. For example, suitable epothilones and epothilone analogs are described in International Publication No. WO 2004/018478 (hereby incorporated by reference). Taxoids, such as paclitaxel and docetaxel, as well as the analogs of paclitaxel taught by U.S. Pat. Nos. 6,610,860; 5,530,020; and 5,912,264 (each incorporated herein by reference) can be used.

Suitable DNA and/or RNA transcription regulators, including, without limitation, actinomycin D, daunorubicin, doxorubicin and derivatives and analogs thereof also are suitable for use in combination with darifenacin therapy.

DNA intercalators and cross-linking agents that can be administered to a subject include, without limitation, cisplatin, carboplatin, oxaliplatin, mitomycins, such as mitomycin C, bleomycin, chlorambucil, cyclophosphamide and derivatives and analogs thereof.

DNA synthesis inhibitors suitable for use as therapeutic agents include, without limitation, methotrexate, 5-fluoro-5′-deoxyuridine, 5-fluorouracil and analogs thereof.

Examples of suitable enzyme inhibitors include, without limitation, camptothecin, etoposide, formestane, trichostatin and derivatives and analogs thereof.

Suitable compounds that affect gene regulation include agents that result in increased or decreased expression of one or more genes, such as raloxifene, 5-azacytidine, 5-aza-2′-deoxycytidine, tamoxifen, 4-hydroxytamoxifen, mifepristone and derivatives and analogs thereof.

“Angiogenesis-inhibitors” include molecules, such as proteins, enzymes, polysaccharides, oligonucleotides, DNA, RNA, and recombinant vectors, and small molecules that function to reduce or even inhibit blood vessel growth. Angiogenesis is implicated in most types of hu-man solid tumors. Angiogenesis inhibitors are known in the art and examples of suitable angiogenesis inhibitors include, without limitation, angiostatin K1-3, staurosporine, genistein, fumagillin, medroxyprogesterone, suramin, interferon-alpha, metalloproteinase inhibitors, platelet factor 4, somatostatin, thrombospondin, endostatin, thalidomide, and derivatives and analogs thereof.

Kinase inhibitors include Gleevac, Iressa, and Tarceva that prevent phosphorylation and activation of growth factors.

Antibodies that can be used include Herceptin and Avastin that block growth factors and the angiogenic pathway.

Other therapeutic agents, for example anti-tumor agents, that may or may not fall under one or more of the classifications above, also are suitable for administration in combination with darifenacin (or other M3 mAChR antagonist) therapy. By way of example, such agents include adriamycin, apigenin, rapamycin, zebularine, cimetidine, and derivatives and analogs thereof.

“Nicotinic receptor antagonists” include compounds that significantly reduce the activity of nicotinic receptors. Particular examples of such agents that can be used in combination which darifenacin or other M3 mAChR antagonist therapy (for example in a single therapeutic composition or administered separately), include, but are not limited to: Inversine® (mecamylamine HCl), methyllycaconitine, and dihydro-β-erythrodine, as well as pharmaceutically acceptable derivatives thereof. For example, mecamylamine HCl can be administered to a subject having or at risk for developing a tumor that expresses both M3 mAChRs and nicotinic receptors in combination with darifenacin (or other M3 mAChR antagonist). Mecamylamine HCl has been used for the treatment of hypertension. In one example, a subject is administered mecamylamine HCl at a total daily dosage of 2.5-75 mg (such as 2.5-25 mg), for example in 1 to 4 divided doses (such as in 1, 2, 3 or 4 doses). In a particular example, mecamylamine HCl is administered orally at a dosage of 2.5 mg twice a day, up to 25 mg three times a day. If desired, a composition that includes both mecamylamine HCl and darifenacin (or other M3 mAChR antagonist) at the desired dosage can be administered orally to the subject.

In one example, at least a portion of the tumor (such as a tumor that expresses M3 mAChRs) is surgically removed, irradiated, or both, prior to administration of darifenacin or other M3 mAChR-antagonist. For example, a subject having a lung cancer or other solid tumor can have the tumor surgically excised prior to administration of darifenacin (or other M3 mAChR antagonist). In another particular example, the subject has a tumor and is administered radiation therapy prior to administration of darifenacin or other M3 mAChR antagonist.

### Evaluating Expression

In one example, the method further includes determining whether a sample obtained from the tumor expresses M3 mAChRs, nicotinic receptors, choline acetyltransferase (ChAT), or combinations thereof. For example, using routine methods M3 mAChR, nicotinic receptor, or ChAT proteins or nucleic acids (such as DNA or RNA) can be detected. In some examples, the level of M3 mAChR, nicotinic receptor, or ChAT is quantitated. For example, the relative or absolute quantity of M3 mAChR, nicotinic receptor, or ChAT in a sample can be quantitated.

Methods of obtaining a biological sample from a subject are known in the art. For example, a sample from the tumor that contains cellular material can be obtained by surgical excision of all or part of the tumor, by collecting a fine needle aspirate from the tumor, as well as other methods known in the art. If desired, the sample can be concentrated or purified before use. For example, proteins or nucleic acids can be isolated from the sample. Alternatively, the sample can be used directly.

In particular examples, a tumor sample obtained from the subject is analyzed to determine if it contains detectable levels of M3 mAChR, nicotinic receptor, or ChAT proteins. For example immunohistochemistry using antibodies to the desired protein can be performed on tissue sections from formalin-fixed, paraffin embedded tumor samples or on frozen sections of tumor samples. Methods of demonstrating that a tumor cell (such as a cancer cell) expresses one or more particular proteins are known in the art. For example, Blanco and Robinson teach a method for immunostaining paraffin embedded tissues to detect M3 mAChR (Ann. Diagn. Pathol. 8:333-6, 2004, herein incorporated by reference as to the method). de la Torre et al. teach a method for detecting M3 mAChR in mammary adenocarcinomas using Western blotting (Breast Cancer Res. 7:R345-52, 2005; herein incorporated by reference as to the method). In addition, Plummer et al. teach a method for detecting alpha-7 nicotinic receptors in human lung cancer cells using Western blotting (Respir. Res. 6:29, 2005; herein incorporated by reference as to the method). An exemplary method for detecting CHAT proteins in human cancer cells is provided in Mellott et al. (Eur. J. Biochem. 269:850-8, 2002).

For example, immunoassays can be used to detect the presence of M3 mAChR, ChAT, or nicotinic receptor proteins in the sample. Generally, immunoassays include the use of one or more specific binding agents (such as antibodies) that can substantially only bind to M3 mAChR, CHAT, or nicotinic receptor. Such binding agents can include a detectable label (such as a radiolabel, fluorophore or enzyme), that permits detection of the binding to the protein. Exemplary immunoassays that can be used include, but are not limited to: Western blotting, ELISA, fluorescence microscopy, and flow cytometry. The presence of detectable signal above background or control levels indicates that the tumor expresses M3 mAChR, ChAT, or nicotinic receptor proteins.

In particular examples, a tumor sample obtained from the subject is analyzed to determine if it contains detectable levels of M3′ mAChR, ChAT, or nicotinic receptor nucleic acid molecules, such as cDNA or mRNA. For example, assays that permit detection of nucleic acids can be used. Exemplary assays that can be used include, but are not limited to: Northern blotting, Southern blotting, and PCR (such as RT-PCR). In one example, a nucleic acid probe that hybridizes to an M3 mAChR, ChAT, or a nicotinic receptor nucleic acid is contacted with the sample. For example, the probe can be incubated with the sample under high stringency conditions (such as when the hybridization is performed at about 42° C. in a hybridization solution containing 25 mM KPO₄ (pH 7.4), 5×SSC, 5× Denhart's solution, 50 μg/mL denatured, sonicated salmon sperm DNA, 50% formamide, 10% Dextran sulfate, and 1-15 ng/mL probe (about 5×10⁷ cpm/μg), while the washes are performed at about 65° C. with a wash solution containing 0.2×SSC and 0.1% sodium dodecyl sulfate), wherein the presence of detectable signal from the probe above background or control levels indicates that the tumor includes M3 mAChR, ChAT, or nicotinic receptor nucleic acid molecules.

In another example expression of acetylcholine by the tumor can be determined by performing immunohistochemistry for choline acetyltransferase or measuring the acetylcholine content of the tumor by HPLC (high performance liquid chromatography).

### Therapeutic Compositions

Another aspect of the disclosure includes pharmaceutical compositions prepared for administration to a subject and which include a therapeutically effective amount of one or more of the currently disclosed compounds. For example, compositions that include a therapeutic amount of an M3 muscarinic antagonist, such as darifenacin, can be formulated for use in treating a tumor that expresses M3 mAChRs.

In one example, compositions that include a therapeutic amount of darifenacin (or other M3 mAChR) and one or more additional biologically active agents are provided. Examples of such biologically active agents are described above, and can include chemotherapeutic agents as well as nicotinic receptor antagonists.

In a particular examples, the composition includes 3.75-40 mg darifenacin (for example in a slow-release matrix tablet as described above), and a therapeutically effective amount of an anti-neoplastic chemotherapeutic agent.

In another particular example, the composition includes 3.75 mg-40 mg darifenacin (for example in a slow-release matrix tablet as described above), and a therapeutically effective amount of a nicotinic receptor antagonist (such as mecamylamine HCl). For example, the composition can include 3.75 mg-40 mg darifenacin (for example in a slow-release matrix tablet as described above) and 2.5 mg-75 mg (such as 2.5 mg-25 mg) mecamylamine HCl.

### EXAMPLE 1

### Expression of M3 mAChRs and Choline Acetyltransferase in Tumors

This example describes methods used to determine whether a tumor expressed M3 muscarinic receptors, choline acetyltransferase, or both. Although particular immunohistochemical methods are described, one skilled in the art will appreciate that other methods of detection can be used.

A panel of archival specimens was screened for coexpression of M3 mAChR and choline acetyltransferase (ChAT), the enzyme necessary for ACh synthesis. Paraffin embedded sections (5 μm) of SCLC and other tumor types were cut from tissue blocks and stained with H&E to confirm diagnosis and tissue integrity. Serial sections were processed for immunohistochemistry using known methods (Song et al. Cancer Res. 63:214-21, 2003). Antibodies used were mouse anti-choline acetyltransferase (mAB 305, Chemicon International, Inc, 1:400) and rabbit anti M3 mAChR (H210, Santa Cruz Biotechnology, Inc., Santa Cruz, Calif.). All analyses also included non-immune serum controls. Intensity of immunohistochemical staining was scored from 0 to 4+ (where 0=no staining, 1+=focal weak staining, 2+=focal strong staining or diffuse weak staining, 3+=diffuse medium staining, 4+=diffuse strong staining). Dual immunohistochemical staining was performed using the same primary antibodies and donkey anti-mouse Alexa-488 and donkey anti-rabbit Alexa-594 labeled second antibodies from Molecular Probes; Inc. (Eugene, Oreg.).

Table 1 shows the frequency with which several cancer types express both M3 mAChRs and choline acetyltransferase (ChAT). In a series of 24 SCLC tumors, 17 of 24 (700%) tumors expressed M3 mAChR immunoreactivity with an average intensity of 1.2 of 4. In addition all 17 SCLC that expressed M3 also expressed CHAT with average immunostaining intensity of 1.4 of 4 (Table 1). Thus 70% of the SCLC screened expressed both ChAT and M3R, indicating that M3 receptor antagonists can significantly reduce the growth of the majority of SCLC. In addition, the widespread expression of ACh and M3 mAChRs in non-neuronal cells indicates that the autocrine cholinergic loop can occur in other tumor types besides SCLC. Tumors that express both M3 receptors and ChAT can have a therapeutic response to M3 mAChR antagonists, and in some examples a greater therapeutic response than expression of a tumor that expresses M3 mAChR but not ChAT.

TABLE 1
Frequency of ChAT and M3 mAChR coexpression in selected cancers.*

| | | | | % M3 and ChAT |
|---|---|---|---|---|
| Cancer type | N | % M3 | % ChAT | coexpression |
| Lung (SCLC) | 24 | 70 | 92 | 70 |
| Lung (BAC) | 20 | 85 | 80 | 70 |
| Lung (SCC) | 31 | 71 | 58 | 45 |
| Pancreatic | 32 | 78.1 | 65.6 | 50 |
| Cervical | 14 | 50 | 71 | 43 |

*Frequency of M3 mAChR, ChAT and their coexpression in archival samples of SCLC, bronchoalveolar lung carcinoma (BAC), squamous cell lung carcinoma (SCC), pancreatic carcinoma and cervical carcinoma as determined by immunostaining. Sample size of each series as shown (N).

### EXAMPLE 2

### Darifenacin Decreases SCLC Growth In Vitro

This example describes methods used to demonstrate that treatment with therapeutic amounts of darifenacin in vitro can decrease, growth of SCLC cells. One skilled in the art will appreciate that similar in vitro methods can be used to determine if darifenacin decreases tumor growth in vitro.

SCLC cell lines (HA-E, SV-E, LV-E, BK-T, HG-E, AD-A, LD-T, H69 (HTB-119), H82 (HTB-175), H146 (HTB 173), H187 (CRL-5804), H524 (CRL-5831), H526 (CRL-5811), H740 (CRL-5840), H841 (CRL-5845), H1048 (CRL-5853), H1105 (CRL-5856), H1417 (CRL-5869), H1694 (CRL-5888). H1836 (CRL-5898), H1876 (CRL-5902), H2029 (CRL-5913), H2195 (CRL-5931), and H2198 (CRL-5933)), were grown in RPMI 1640 medium (Mediatech, Herndon Va.) supplemented with 0-10% fetal bovine serum (Hyclone, Logan, Utah) and ITS (5 μg/ml insulin, 5 μg/ml transferrin, 5 ng/ml sodium selenite (BD Biosciences, San Jose, Calif.)) plus 100 units/ml penicillin, 100 mg/ml streptomycin, and 2.5 mg/ml amphotericin (Invitrogen, Carlsbad Calif.) at 37° C. in a humidified 5% CO₂ atmosphere.

For in vitro cell proliferation assays, cells were suspended at a concentration of 50,000 cells/ml and pipetted up and down to break up cell clumps. 100 μl cell suspension was added to each well of a 96-well plate. 100 μl darifenacin at concentrations of 2×10⁻⁵, 2×10⁻⁶, 2×10⁻⁷, 2×10⁻⁸, 2×10⁻⁹ M and vehicle control were then added to wells. Every three days, half the volume of the media was changed and at 0, 3, 6, 9 and 12 days, cell number was determined with the Cell-Titer Blue reagent (Promega, Madison Wis.) which measures the production of the fluorescent product of resorufin from resazurin by metabolically active cells.

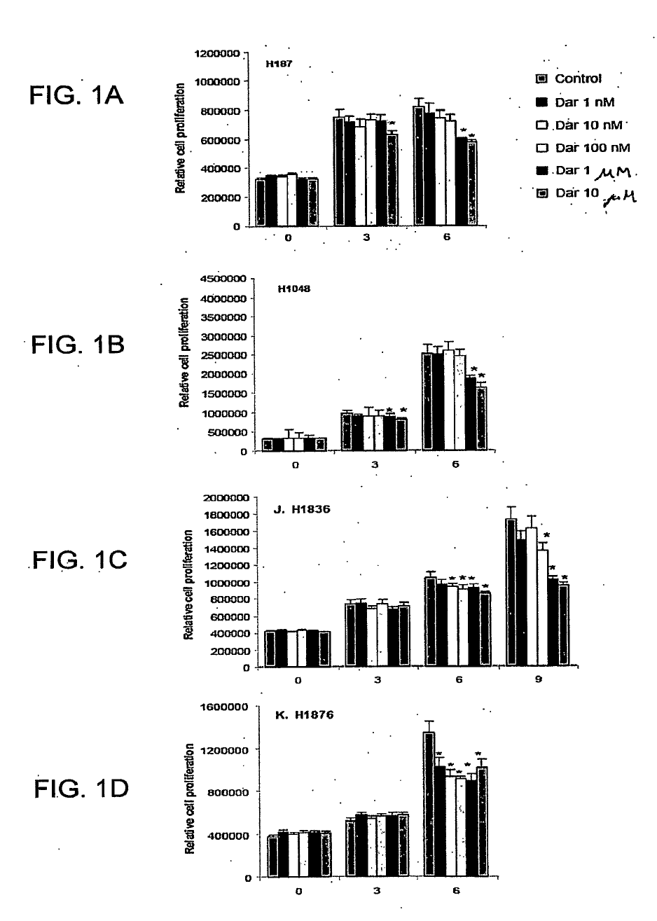

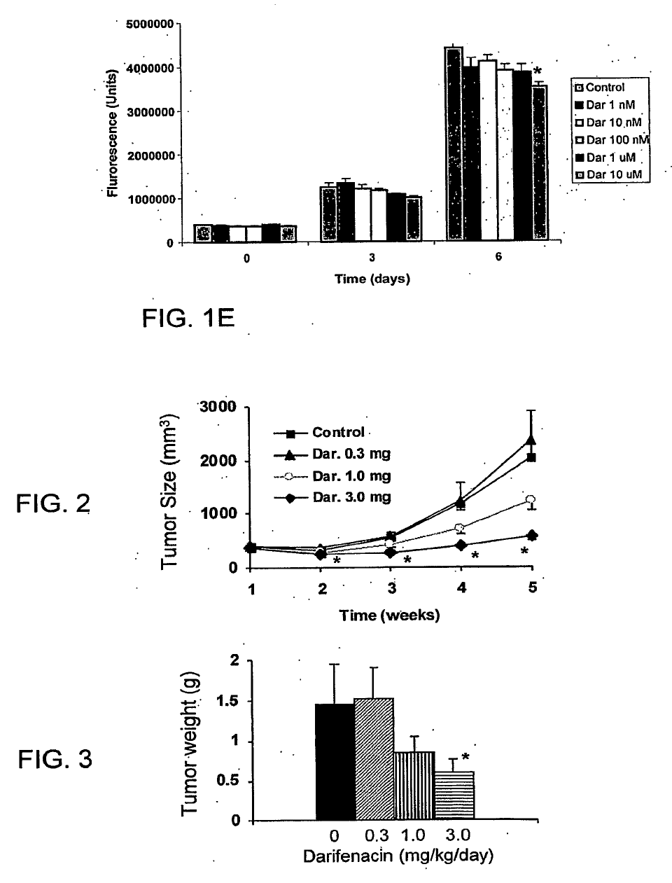

FIGS. 1A-E shows the effect of darifenacin reducing cell growth in five SCLC cell lines. The concentration of darifenacin needed to achieve this result depended on the cell line, though all five cell lines were responsive to 10 μM darifenacin. Darifenacin significantly reduced proliferation in 14 out of 20 SCLC cell lines tested. Cell lines LV-E, AD-A, L-D-T, H69, H1417, and H2198 were not affected.

Similarly, treatment of the SCLC cell line H82 with 10⁻⁹ mol/L to 10⁻⁵ mol/L of the M3 mAChR antagonist 4-DAMP or 10⁻⁵ mol/L of the M3 mAChR antagonist P-F-HHSiD significantly reduced cell proliferation after nine days. In contrast, treatment of H82 cells with up to 10⁻⁵ mol/L pirenzepine (M1-selective mAChR antagonist) or up to 10⁻⁵ mol/L of the M2/M4-selective mAChR antagonist AFDX-1116 had no detectable effect on H82 cell proliferation, even after nine days of treatment.

### EXAMPLE 3

### Treatment of Lung Cancer in Mice Using Subcutaneous Darifenacin

This example describes methods used to reduce the growth of human lung cancer cells in mice, by subcutaneous administration of darifenacin. One skilled in the art will appreciate, based on these teachings, that similar methods can be used to treat other mammals, such as humans, as well as other types of tumors (such as a tumor that expresses M3 muscarinic receptors).

Mice (NU/NU, Charles River Laboratories) were injected with SCLC tumor cells (NCI-H82 cells, American Type Culture Collection (ATCC); Manassas, Va.), treated with drug and tumor growth monitored. The H82 cells express M3 muscarininc receptor (see Song et al., Cancer Res. 63:214-21, 2003). Nude mice, 5-7 weeks old were housed in a pathogen-free room in microisolators with autoclaved bedding, and fed autoclaved rodent chow and water.

H82 cells were first grown in RPMI-1640+ITS (insulin (5 μg/ml), transferrin (5 μg/ml), sodium selenite (5 ng/ml)). Cells were collected, centrifuged and resuspended in fresh RPMI-1640+ITS at a concentration of 5×10⁶ cell/0.25 ml. Cells were then mixed with an equal volume of matrigel (BD Biosciences) to give a final concentration of 5×10⁶ cell/0.5 ml and 0.5 ml of this cell suspension was then injected subcutaneously into the right flank of each mouse.

Tumors were allowed to grow for one week, then drug administration was initiated and continued for the next 4 weeks. Drugs were administered by subcutaneously-implanted osmotic minipumps (Alzet model# 2004). Atropine and darifenacin were dissolved in 50% DMSO/50% phosphate-buffered saline. Atropine was delivered at a dose of 10 mg/kg per day; darifenacin (Novartis Pharma AG, Basel, Switzerland) was delivered at doses of 0.3, 1.0 and 3.0 mg/kg per day. Control animals received minipumps filled with 50% DMSO/50% phosphate-buffered saline.

Tumor volume was determined weekly by measuring with calipers (volume=height×width×depth). Mice were euthanized after 4 weeks of drug administration. 10 animals were used per group. At sacrifice tumors were removed and weighed. Part of the tumor was fixed for histologic examination of tumor morphology and part was frozen for RNA and protein analyses.

As shown in FIG. 2, consistent with the in vitro effects of M3 mAChR antagonists on tumor cell growth, darifenacin caused a dosewise-reduction in tumor size that continued for the entire time period and for which the inhibition for the largest dose was statistically significant. In addition as shown in FIG. 3 darifenacin caused a significant decrease in tumor weight (as determined by removing and weighing the tumors). No significant effects of darifenacin treatment on overall animal weight were observed. These results demonstrate that tumor secretion of ACh in vivo can stimulate SCLC tumor growth and that M3 mAChR antagonists can block autocrine cholinergic stimulation of tumor growth.

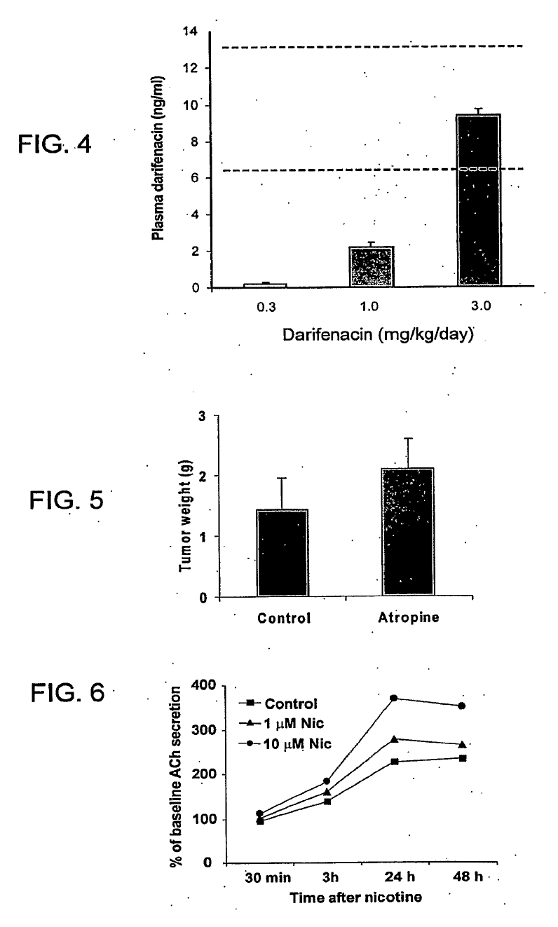

Inhibition of tumor growth was achieved at plasma darifenacin concentrations between 3 and 10 ng/ml (average 9.4±0.3 ng/ml, ˜2.5×10⁻⁸ M) darfenacin that is commonly obtained when darifenacin is used clinically) which are in the range of plasma concentration obtained with clinical use of darifenacin for control of overactive bladder in humans (FIG. 4). Thus significant reduction in SCLC growth can be achieved at clinically relevant doses of M3 mAChR antagonists.

Levels of MAPK phosphorylation in the tumors removed from the darifenacin-treated mice were significantly decreased, while no significant effects were seen on Akt phosphorylation. ACh content of control tumor samples was 420±110 pmol/g which, corresponds to 4×10⁻⁷ M, assuming uniform distribution in the tumor. These levels of ACh are high enough to increase MAPK phosphorylation. This indicates that autocrine ACh signals proliferation primarily through the MAPK pathway and that other factors stimulate Akt phosphorylation. This indicates that a combination regimen of Akt inhibitors (for example Perifosine which can be administered orally 100 mg p.o.×four doses (every 6 hours) followed by a 50 mg p.o., once daily as described in Van Ummersen et al., Clin. Cancer Res., 10:7450-6, 2004) and M3 receptor antagonists may be effective in inhibiting tumor growth, for example in smokers in whom nicotine has activated Akt proliferative pathways. Histologically, cells in tumors from nude mice treated with the highest dose of darifenacin were larger in size, with increased-cytoplasm and less nuclear molding as compared to controls consistent with slower proliferation.

As shown in FIG. 5 the muscarinic antagonist is selective for M3. Nonselective antagonists such as atropine or trospium stimulate all subtypes of muscarinic receptors equally and do not reduce or inhibit tumor growth. These, results demonstrate that darifenacin can be used to treat tumors in a mammal.

### EXAMPLE 4

### Treatment of Lung Cancer in Mice Using Oral Darifenacin

This example describes methods that can be used to reduce the growth of human lung cancer cells in mice, by oral administration of darifenacin. One skilled in the art will appreciate, based on these teachings, that similar methods can be used to treat other mammals, such as humans, as well as other types of tumors (such as a tumor that expresses M3 muscarinic receptors).

As shown in FIGS. 2 and 3, darifenacin administered subcutaneously treats the lung cancer cells. Darifenacin has excellent bioavailability and therefore will be highly effective given orally to reduce or inhibit tumor growth in nude mice.

Nude mice are administered H82 cells as described in Example 3. One week later, oral administration of darifenacin by oral gavage once daily is initiated. Darifenacin is delivered by oral gavage at concentrations of 0.03, 0.1, 0.3, 1, 3, and 10 mg/kg per day prepared in 0.1 ml polyethylene glycol-400 for four weeks. Control animals will receive 0.1 ml polyethylene glycol-400 by oral gavage.

Tumor volume will be determined weekly as described above, and mice euthanized after 4 weeks of drug administration. At sacrifice tumors are removed and weighed. Part of the tumor can be fixed for histologic examination of tumor morphology and part frozen for RNA and protein analyses.

It is expected that mice receiving darifenacin will have smaller tumors (or even completely eliminated tumors) as compared to the control mice.

### EXAMPLE 5

### Treatment of Lung Cancer in Mice Using Darifenacin and Mecamylamine

This example describes methods that can be used to reduce the growth of human lung cancer cells in mice, by administration of darifenacin and a nicotine receptor antagonist (such as mecamylamine). Both subcutaneous and oral administrations are described. Oral forms of mecamylamine are commercially available as Inversine® from Targacept, Inc. (Winston-Salem, N.C.). One skilled in the art will appreciate, based on these teachings, that similar methods can be used to treat other mammals, such as humans having or at risk for developing tumors that express M3 muscarinic and nicotinic receptors.

Nude mice are administered H82 cells as described in Example 3. H82 cells express both M3 muscarinic and nicotinic receptors. One week later, administration of darifenacin and mecamylamine is initiated.

For subcutaneous-administration of the darfenacin and mecamylamine, the methods described in Example 3 are used, except minipumps containing darifenacin will also include mecamylamine to deliver 0.1, 0.3, 1 and 3.0 mg/kg per day. Mecamylamine (Sigma Chemical) is dissolved in 50%. DMSO for darifenacin.

For oral administration of the darifenacin and mecamylamine, the methods described in Example 4 are used, except that 0.1, 0.3, 1 and 3.0 mg mecamylamine/kg per day will be orally administered in addition to the darifenacin.

Tumor volume will be determined weekly as described in the Examples above, and mice euthanized after 4 weeks of drug administration. At sacrifice tumors are removed and weighed. Part of the tumor can be fixed for histologic examination of tumor morphology and part frozen for RNA and protein analyses.

It is expected that mice receiving darifenacin and mecamylamine will have smaller tumors (or even completely eliminated tumors) as compared to the control mice, and may demonstrate an enhanced effect when both agents are used together (for example as compared to each alone).

Using these methods, an optimum dose of mecamylamine to reduce or inhibit tumor growth is determined. Using the methods described above, mice can be administered the optimum dose of mecamylamine combined with the optimum dose of darifenacin to reduce or inhibit tumor growth in nude mice.

### EXAMPLE 6

### Treatment of Lung Cancer in Mice Receiving Nicotine

This example describes methods that can be used to demonstrate that darifenacin can oppose the ability of nicotine to stimulate lung cancer growth. Such methods can be used to demonstrate the effectiveness of darifenacin to reduce or inhibit tumor growth in cancer patients who continue to smoke.

Nude mice are administered H82 cells as described in Example 3. One week later, administration of darifenacin (alone or in combination with mecamylamine) is initiated orally or subcutaneously as described in Examples 3 and 4. Nicotine (nicotine bitartrate, Sigma) is dissolved in water at a concentration to deliver 2.0 mg/kg/day; and will be administered by a second minipump. Tumor volume will be determined weekly as described in the Examples above, and mice euthanized after 4 weeks of drug administration. At sacrifice tumors are removed and weighed. Part of the tumor can be fixed for histologic examination of tumor morphology and part frozen for RNA and protein analyses.

It is expected that darifenacin will be able to reduce tumor growth in mice also receiving nicotine, as compared to controls not receiving darifenacin. Nicotine will likely cause the tumors to grow faster because as shown in FIG. 6, nicotine stimulates acetylcholine secretion from lung cancer cells.

### EXAMPLE 7

### Decreasing Cell Proliferation in Mice Induced by Nicotine

This example describes methods that can be used to decrease (for example prevent) proliferation of normal cells induced by nicotine. Stimulation of cell growth may increase the risk of developing cancer and reducing or preventing this increase may lessen the risk of developing cancer in smokers.

Mice will be treated with nicotine or water for 3 weeks as described in Example 6 above. At the end of 3 weeks, bromodeoxy uridine (BrdU) will be injected ip, 50 mg/kg, then 24 hours later mice will be sacrificed and selected tissues removed to measure cell proliferation. Tissues removed will include lung, pancreas and esophagus, tumors of which are associated with smoking. Tissues will be sectioned and BrdU incorporation into cells determined so as to determine the number of replicating cells per section.

It is expected that there will be more replicating cells in tissue removed from nicotine-treated mice than control mice. Additional mice will be treated with nicotine plus M3 antagonists as described in Examples 3 and 4. It is expected that the M3 antagonist will block the nicotine-induced increase in cell proliferation.

### EXAMPLE 8

### Reducing Smoking-Induced Cancer

This example describes methods that can be used to decrease (for example prevent) proliferation of normal cells induced by nicotine.

The A/J strain of mice exposed to tobacco smoke for 5 months develop spontaneous lung tumors. A/J mice will be exposed to tobacco smoke for 5 months (for example using the methods described in Witschi et al., Inhal. Toxicol. 16:27-32, 2004). The exposed mice will be treated with vehicle or M3 antagonist and development of lung cancers quantified by immunohistochemical analysis of the lungs using tumor specific antibodies.

It is expected that administration of an M3 antagonist will decrease the development of lung cancers in the mice exposed to tobacco smoke.

### EXAMPLE 9

### Treatment of Other Cancers in Mice

This example describes methods than can be used to demonstrate that darifenacin can also be used to treat other tumors in vivo, such as tumors that express M3 muscarinic receptors.

Nude mice are administered tumor cells as described in Example 3. Exemplary cells include squamous cell lung carcinoma cells, bronchoalveolar lung carcinoma cells, as well as cancer cells from tissues other than lung. Examples of squambus cell carcinomas are H520 and H1385 cells from ATCC. Examples of bronchoalveolar cells are A549 cells from ATCC. Pancreatic carcinoma cells are also available from ATCC.

One week later, administration of darifenacin (alone or in combination with mecamylamine) is initiated orally or subcutaneously as described in Examples 3 and 4. Tumor volume will be determined weekly as described in the Examples above, and mice euthanized after 4 weeks of drug administration. At sacrifice tumors are removed and weighed. Part of the tumor can be fixed for histologic examination of tumor morphology and part frozen for RNA and protein analyses.

It is expected that darifenacin will also be able to reduce or inhibit the growth of other tumors associated with smoking, such as a reduction of at least 10%, at least 20% at least 50%, at least 80%, at least 90%, or even 100% as compared to subjects not receiving darifenacin.

### EXAMPLE 10

### Treatment of Tumors in Humans

This example describes methods that can be used to reduce the growth of a tumor in humans by oral administration of darifenacin, for example treatment of tumor cells that express M3 muscarinic receptors. Exemplary tumors that express M3 muscarinic receptors include but are not limited to, lung cancer, pancreatic cancer, cervical cancer, colon cancer, brain cancer, and ovarian cancer. Although particular methods, dosages, and modes of administrations are provided, one skilled in the art will appreciate that variations can be made without substantially affecting the treatment.

The results in mice demonstrated that tumor growth was significantly reduced by a plasma concentration of darifenacin of 9.4±0.97 ng/ml. Clinical data shows that humans taking 30 mg of darifenacin per day in the form of slow release tablets (Enablex, Novartis) have average minimum and maximum plasma concentrations of between 6.4 and 13.2 ng/ml. Therefore human patients are treated with 30 mg-darifenacin given as two 15 mg extended release tablets taken orally once daily, for example for a period of at least 6 months, at least one year, at least 2 years, or at least five years.

Darifenacin can be used in conjunction with other cancer therapy (for example rather than replacing the therapy). Thus darifenacin can be added to the usual and customary chemotherapy, surgery and/or radiation treatments' conventionally used for the particular tumor type. Administration of darifenacin can be continued after chemotherapy and radiation therapy was stopped and can be taken long term (for example over a period of months or years).

Briefly, the method can include screening subjects to determine if they have a tumor that expresses M3 muscarinic receptors (or has an increased risk of developing such a tumor). Subjects having a tumor that expresses M3 muscarinic receptors (or are at an increased risk of developing such a tumor) are selected. In a clinical trial, half of the subjects would follow the established protocol for treatment of the tumor (such as a normal chemotherapy/radiotherapy regimen). The other half would follow the established protocol for treatment of the tumor (such as a normal chemotherapy/radiotherapy regimen) in combination with darifenacin. In some examples, the tumor is surgically excised prior to treatment with darifenacin.

In particular examples, the subjects are also screened to determine if their tumor that expresses M3 muscarinic receptors also expresses nicotinic receptors. Tumors can also be screened for expression of choline-acetyltransferase (ChAT). Subjects having a tumor that expresses both M3 muscarinic and nicotinic receptors (or are at an increased risk of developing such a tumor) are selected. Such subjects can additionally receive the nicotinic receptor antagonist mecamylamine (such as Inversine®, Merck), in combination with the established protocol for treatment of the tumor (such as a normal chemotherapy/radiotherapy regimen). Thus, human subjects can orally receive 30 mg darifenacin once per day combined with mecamylamine (5-10 mg twice per day). The optimal dose of mecamylamine can be determined in laboratory animals as described above in Example 5.

### Screening Subjects

In particular examples, the subject is first screened to determine if their tumor expresses M3 muscarinic receptor. For example, a tumor, such as a cancer of the lung, pancreas, or cervix can be screened using routine methods for the presence of M3 muscarinic receptors. The disclosure is not limited to particular screening methods. In particular examples, method further includes quantitating the relative, amount of M3 muscarinic receptors (or other receptors) present. In particular methods, the subject is also screened to determine if their tumor expresses nicotinic receptor. In some methods the subjects can be screened to determine if their tumors express choline acetyltransferase. However, in particular examples, such pre-screening is not required prior to administration of the therapeutic compositions disclosed herein (such as those that include darifenacin).

In one example, the tumor (or a portion thereof, such as a fine needle aspirate or other biopsy sample) is analyzed using immunodetection methods. For example, the biological sample can be incubated with an antibody that specifically binds to a M3 muscarinic receptor or a nicotinic receptor. Such antibodies are commercially available (for example M3 muscarinic receptor antibodies are available from OriGene, Rockville, Md. [catalog number TA200008] and Accurate Chemical, Westbury, N.Y. [catalog number ALAMR006] and nicotinic receptor antibodies are available from FabGennix, Frisco, Tex. [catalog numbers NACR-100P and NACR-101AP] and RDI Division of Fitzgerald Industries Intl., Concord, Mass. [catalog number RDI-NARA4abm-A4]). The primary antibody can include a detectable label. For example, the primary antibody can be directly labeled, or the sample can be subsequently incubated with a secondary antibody that is labeled (for example with a fluorescent label). The label can then be detected, for example by microscopy, ELISA, flow cytometery, or spectrophotometry. Using routine immunohistochemistry methods, M3 mAChR and choline acetyltransferase were detected in SCLC and pancreatic cancer (see Table 1). The M3 mAChR and choline acetyltransferase were detected in a high grade cancer of the cervix. In another example, the biological sample is analyzed by Western blotting for the presence of M3 muscarinic receptor or nicotinic receptor proteins.

As an alternative to analyzing the sample for the presence of proteins, the presence of nucleic acids can be determined. For example, the biological sample can be incubated with primers that permit the amplification of the M3 muscarinic receptor or the nicotinic receptor, under conditions sufficient to permit amplification of the M3 muscarinic receptor or the nicotinic receptor. Exemplary methods include PCR and RT-PCR. In another example, the biological sample is incubated with probes that can bind to an M3 muscarinic receptor or a nicotinic receptor nucleic acid (such as cDNA, genomic DNA, or RNA (such as mRNA)) under high stringency conditions. The resulting hybridization can then be detected using methods known in the art.

### Pre-Treatment of Subjects

In particular examples, the subject is treated prior to administration of a therapeutic composition that includes darifenacin. However, such pretreatment is not always required, and can be determined by a skilled clinician. For example, the tumor can be surgically excised (in total or in part) prior to administration of darifenacin (or both darifenacin and mecamylamine). In addition, the subject can be treated with an established protocol for treatment of the particular tumor present (such as a normal chemotherapy/radiotherapy regimen).

### Administration of Therapeutic Compositions

Administration can be achieved by any method known in the art, such as oral administration, inhalation, or inoculation (such as intramuscular, ip, intravenous, or subcutaneous). In some examples, the therapeutic composition includes darifenacin. In particular examples, darifenacin is administered in combination with other agents, such as mecamylamine.

The amount of darifenacin administered is sufficient to treat a subject having an M3 muscarinic receptor expressing tumor. Similarly, the amount of darifenacin and mecamylamine administered is sufficient to treat a subject having a tumor that expresses M3 muscarinic and nicotinic receptors. An effective amount can being readily determined by one skilled in the art, for example using routine trials establishing dose response curves. In addition, particular exemplary dosages are provided above. The therapeutic compositions can be administered in a single dose delivery, via continuous delivery over an extended time period, in a repeated administration protocol (for example, by a, daily, weekly, or monthly repeated administration protocol).

In one example, therapeutic compositions that include darifenacin or mecamylamine are administered orally to a human. As such, these compositions may be formulated with an inert diluent or with an assimilable edible carrier, or they may be enclosed in hard- or soft-shell gelatin capsule, or they may be compressed into tablets, or incorporated directly with the food of the diet.

The compositions that include darifenacin or mecamylamine (or both) can be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each incorporated herein by reference in its entirety). The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it can contain, in addition to materials of the above type, a liquid carrier. Various other materials may be present as coatings or to otherwise modify the physical form of the dosage unit. For instance, tablets, pills, or capsules may be coated with shellac, sugar, or both. A syrup of elixir can contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds can be incorporated into sustained-release preparation and formulations.

### Assessment:

Following the administration of one or more therapies, subjects having a tumor (for example a tumor that expresses M3 muscarinic receptors alone or in combination with nicotinic receptors) can be monitored for tumor treatment, such as regression or reduction in metastatic lesions. In particular examples, subjects are analyzed one or more times, starting 7 days following treatment

Subjects can be monitored using any method known in the art. For example, diagnostic imaging can be used (such as x-rays, CT scans, MRIs, fiberoptic examination, and laparoscopic examination), as well as analysis of biological samples from the subject (for example analysis of blood, sputum, urine, or other biological samples), such as analysis of the type of cells present, or analysis for a particular tumor marker. In one example, if the subject has lung cancer, assessment can be made using chest x-rays, MRI, or CAT scans, analysis of the type of cells contained in sputum or pulmonary flushings, and fiberoptic examination of the bronchial passages.

### EXAMPLE 11

### Treatment of Smokers

This example describes methods that can be used to treat a smoker who has an increased risk for developing a tumor, such as a tumor that expresses M3 muscarinic receptors. For example such methods can be used to prevent or delay the onset of tumors.

Because normal bronchial epithelium expresses M3 muscarinic receptors and nicotine stimulates acetylcholine secretion (FIG. 6), smokers are at greater risk of developing lung cancer. Therefore smokers clinically defined to be at enhanced risk of developing cancer on the basis of family history, number of pack years smoked or presence of precancerous lesions as determined by sputum analysis or fiber optic examination with our without biopsy or by other biopsy techniques could take darifenacin for up to 5 years or longer if the increased risks for cancer have not gone away.

In a particular example, a smoker is administered darifenacin with 30 mg darifenacin daily (administered as two 15 mg extended release tablets taken once daily, such as those described in U.S. Pat. No. 6,106,864) for at least 5 years.

### EXAMPLE 12

### Use of RNAi Molecules

This example describes the use of RNAi molecules specific for the M3 mAChR. Such RNAi molecules can be used instead of, or in addition to, the M3 mAChR antagonists (such as darifenacin) described herein, for example in the treatment of a tumor. An RNAi molecule is a small interfering RNA as is known in the art (see e.g.: U.S. Pat. No. 6,506,559; Milhavet et al., Pharmacological Reviews 55:629-648, 2003; and Gitlin et al., J. Virol. 77:7159-7165, 2003), and includes antisense molecules, siRNAs, and ribozymes specific for the M3 mAChR, which reduce or prevent expression of M3 mAChR, for example by at least 50%, at least 60%, at least 75%, or at least 90%. In some examples, RNAi molecules are about 19-30 nucleotides in length, such as at least 21 nucleotides, for example at least 23 nucleotides (for example 19, 20, 21, 22, 23, 24, 25, 26, 27, 28, 29, or 30 nucleotides).

The role of M3 receptors in mediating the effect of ACh on [Ca²⁺]ₗ was confirmed by siRNA knockdown as follows. ON-TARGET plus siRNAs for the muscarinic receptor subtypes and negative control siRNA were obtained from Dharmacon (Lafayette, Colo.). The siRNAs were transfected into SCLC cell lines at a concentration 100 nMol each with DharmaFECT 1 according to the manufacturer's instruction. 48 h after transfection, the cells were harvested for [Ca²⁺]ₗ, assay, western blotting analysis and preparation of total RNA. For cell proliferation assay, half the media was changed every three days for fresh media also containing the specific or negative control siRNAs and transfection reagent. Transfection of siRNAs against the M1, M3 and M5 muscarinic receptors all knocked down their target RNAs by greater than 60%, but only knockdown of the M3 mAChR RNA blocked the ACh induced increase in [Ca²⁺]ₗ. Western blot analysis confirmed the expression of M3 mAChR in H82 and H1694 cells showing a single band of 70 kDa.

Methods of generating RNAi molecules, and administering them to a mammal in therapeutic amounts, are known in the art. Such methods can be used in place of, or in addition to, those described in the Examples above (for example Examples 10 or 11). The present disclosure is not limited to particular methods of administering RNAi molecules, and such modes can include injection (i.v., i.p., i.m.), topical, oral, inhalation; or other routes known in the art.

In an example, certain RNAi molecules provided by this disclosure are species of siRNAs. One of ordinary skill in the art can readily generate siRNAs which specifically bind to an M3 mAChR nucleic acid sequence. In an example, commercially available kits, such as siRNA molecule synthesizing kits from PROMEGA® (Madison, Wis.) or AMBION® (Austin, Tex.) can be used to synthesize siRNA molecules. In another example, siRNAs are obtained from commercial sources, such as from INVITROGEN® (Carlsbad, Calif.), DHARMACON® (Lafayette, Colo.) or OPENBIOSYSTEMS® (Huntsville, Ala.). Exemplary M3 RNAi molecules can target the human M3 mAChR sequences shown in GenBank Accession Nos. NM_{—}000740 and NM_{—}000741 (sequence available on Mar. 16, 2007), such as GGTCAACAAGCAGCTGAAG-3′ (SEQ ID NO: 1, corresponding to amino acids 280-300) 5′-CCTCTACACCGTGTACATC-3′ (SEQ ID NO: 2, corresponding to amino acids 259-279).

In certain examples, expression vectors are used to express the at least one siRNA molecule. For example, an expression vector can include a nucleic acid sequence encoding at least one siRNA molecule that recognizes an M3 mAChR nucleic acid molecule. In a particular example, the vector contains a sequence encoding both strands of a siRNA molecule comprising a duplex. In another example, the vector also contains a sequence encoding a single nucleic acid molecule that is self-complementary and thus forms a siRNA molecule. Non-limiting examples of such expression vectors are described in Paul et al., Nature Biotech. 19:505, 2002; Miyagishi and Taira, Nature Biotech. 19:497, 2002; Lee et al., Nature Biotech. 19:500, 2002; and Novina et al., Nature Med. online publication Jun. 3, 2003.

In other examples, siRNA molecules include a delivery vehicle, such as liposomes, for administration to a subject, carriers and diluents and their salts, and can be present in pharmaceutical compositions. Nucleic acid molecules can be administered to cells by a variety of methods known to those of skill in the art, such as encapsulation in liposomes, by iontophoresis, or by incorporation into other delivery vehicles, such as hydrogels, cyclodextrins, biodegradable nanocapsules, and bioadhesive microspheres, or by proteinaceous vectors (see, for example, WO 0053722).

In one example, a nucleic acid/vehicle combination is locally delivered by direct injection or by use of an infusion pump. Injection of an M3 mAChR RNAi molecule, whether subcutaneous, i.m., or intradermal, can take place using standard needle and syringe methods, or by needle-free technologies (see for example PCT Publication No. WO-99/31262). Other delivery routes include oral (such as in tablet or pill form), intrathecal, intraperitoneal, or inhalation delivery. More detailed descriptions of nucleic acid delivery and administration are provided in PCT WO 94/02595, PCT WO93/23569, PCT WO99/05094, and PCT WO99/04819.

Alternatively, certain siRNA molecules can be expressed within cells from eukaryotic promoters. Any nucleic acid can be expressed in eukaryotic cells using the appropriate DNA/RNA vector. The activity of such nucleic acids can be augmented by their release from the primary transcript by an enzymatic nucleic acid (PCT WO 93/23569 and PCT WO 94/02595).

In other examples, siRNA molecules can be expressed from transcription units (see for example, Couture et al., 1996, TIG 12:510) inserted into DNA or RNA vectors. The recombinant vectors can be DNA plasmids or viral vectors. siRNA expressing viral vectors can be constructed based on, for example, but not limited to, adeno-associated virus, retrovirus, adenovirus, lentivirus or alphavirus. In another example, pol III based constructs are used to express nucleic acid molecules of the invention (see for example, U.S. Pat. Nos. 5,902,880 and 6,146,886).

The recombinant vectors capable of expressing the siRNA molecules can be delivered as described above, and persist in target cells. Alternatively, viral vectors can be used that provide for transient expression of nucleic acid molecules. Such vectors can be repeatedly administered as necessary. Once expressed, the siRNA molecule interacts with the target M3 mAChR mRNA and generates an RNAi response. Delivery of siRNA molecule expressing vectors can be systemic, such as by i.v. or i.m. administration, by administration to target cells ex-planted from a subject followed by reintroduction into the subject, or by any other means that would allow for introduction into a desired target cell.

In particular examples, a mammal receives an average of 0.5×10⁶ to 1.0×10⁸ molecules of an M3 mAChR RNAi (such as 0.5×10⁶ to 1.0×10⁶ molecules). Numbers of molecules injected per adult are given as rough approximations based on concentration of RNA in the injected material (estimated from ethidium bromide staining) and injection volume (estimated from visible displacement at the site of injection). A variability of several-fold in injection volume between individual subject is possible.

In view of the many possible embodiments to which the principles of the disclosure may be applied, it should be recognized that the illustrated embodiments are only examples and should not be taken as limiting the scope of the disclosure. Rather, the scope of the disclosure is defined by the following claims. We therefore claim as our invention all that comes within the scope and spirit of these claims.

1. A method of treating a tumor, comprising:
administering to the subject a therapeutically effective amount of an M3 muscarinic receptor antagonist, thereby treating the tumor.

2. The method of claim 1, wherein the tumor expresses M3 muscarinic receptors.

3. The method of claim 1, wherein the subject has a tumor that expresses M3 muscarinic receptors.

4. The method of claim 1, wherein the subject has an increased risk for developing a tumor that expresses M3 muscarinic receptors.

5. The method of claim 1, wherein the M3 muscarinic receptor antagonist comprises darifenacin, 4-diphenylacetoxy-N-methylpiperidine methiodide (4-DAMP), hexahydro-sila-difenidol hydrochloride, p-fluoro analog (p-F-HHSiD), solifenacin, zamifenacin, oxybutynin, tiotropium, or a pharmaceutically acceptable derivative thereof.

6. The method of claim 5, wherein the darifenacin is in the form of a pharmaceutically acceptable salt.

7. The method of claim 6, wherein the darifenacin is in the form of its hydrobromide salt.

8. The method of claim 5, wherein the darifenacin or a pharmaceutically acceptable derivative thereof is administered in a dosage form that is adapted to release at least 10% of said darifenacin or the pharmaceutically acceptable derivative thereof in the lower gastrointestinal tract of the subject.

9. The method of claim 8, wherein the dosage form is a slow release matrix tablet.

10. The method of claim 1, wherein administration of the M3 muscarinic receptor antagonist is oral.

11. The method of claim 10, wherein the therapeutically effective amount of darifenacin or a pharmaceutically acceptable derivative thereof comprises 3.75-40 mg daily by oral administration to the subject.

12-13. (canceled)

14. The method of claim 1, wherein the method further comprises analyzing a sample obtained from the tumor to determine whether the tumor expresses M3 muscarinic receptors.

15. The method of claim 1, wherein the method further comprises administering one or more additional therapeutic agents at a therapeutically effective amount to the subject.

16-17. (canceled)

18. The method of claim 2, wherein the tumor that expresses M3 muscarinic receptors further expresses nicotinic receptors, choline acetyltransferase, or both.

19. The method of claim 1, further comprising determining whether the tumor expresses M3 muscarinic receptors, nicotinic receptors, choline acetyltransferase, or combinations thereof.

20. The method of claim 18, further comprising administering to the subject a therapeutically effective amount of a nicotinic receptor antagonist.

21. The method of claim 20, wherein the nicotinic receptor antagonist comprises mecamylamine HCl or a pharmaceutically acceptable derivative thereof.

22. The method of claim 2, wherein the tumor that expresses M3 muscarinic receptor comprises a tumor associated with smoking.

23-25. (canceled)

26. The method of claim 1, wherein the method further comprises surgical excision of the tumor prior to administering the therapeutically effective amount of the M3 muscarinic receptor antagonist.

27. The method of claim 1, wherein treating the tumor comprises reducing growth of cells of the tumor in the subject.

28. The method of claim 1, wherein treating the tumor prolongs survival time of the subject.

29. The method of claim 4, wherein the subject at risk for developing a tumor is a smoker, wherein treating the tumor comprises preventing or delaying the development of a tumor that expresses M3 muscarinic receptor in the subject.

30. (canceled)

31. A composition comprising 3.75-40 mg darifenacin, and a therapeutically effective amount of a nicotinic receptor antagonist or a therapeutically effective amount of an anti-neoplastic chemotherapeutic agent.

32. The composition of claim 31, wherein the therapeutically effective amount of a nicotinic receptor antagonist comprises 2.5 mg-75 mg of mecamylamine HCl.

33-40. (canceled)

41. A method of treating a tumor that expresses M3 muscarinic receptor in a human having said tumor, comprising administering darifenacin, or a pharmaceutically acceptable derivative thereof, to the human in need of such treatment.

42. A method of treating a tumor that expresses choline acetyltransferase or synthesizes acetylcholine and M3 muscarinic receptors, comprising administration of a therapeutically effective amount of a M3 muscarinic receptor antagonist to a subject having the tumor.

43. A method of treating a tumor that expresses M3 muscarinic receptors, wherein the tumor is in a smoker, and wherein the tumor has increased choline acetyltransferase expression or acetylcholine synthesis, comprising:
administration of a therapeutically effective amount of a M3 muscarinic receptor antagonist to the subject having the tumor.

44. A method of treating a smoker who has a greater risk of smoking related cancers due to increased M3 muscarinic receptor expression and acetylcholine synthesis in tissues that are at risk of becoming cancerous, comprising:
administration of a therapeutically effective amount of a M3 muscarinic receptor antagonist to the smoker.

45. A method of treating a tumor, comprising:
administering to the subject a therapeutically effective amount of an M3 muscarinic receptor RNAi molecule, thereby treating the tumor.
